(19) United States
(12) Patent Application Publication
(10) Pub. No.: US 2022/0151491 A1
(43) Pub. Date: May 19, 2022

(54) METHOD AND DEVICE FOR NON-INVASIVE MEASUREMENT OF AN INTRAOCULAR PRESSURE

(71) Applicant: N.M.B. MEDICAL APPLICATIONS LTD., Herzliya (IL)

(72) Inventors: Etai BEYAR, Tel Aviv (IL); Mordechay BEYAR, Tel Aviv (IL); Oren GLOBERMAN, Kfar Shmaryahu (IL); Danielle VALES, Tel Aviv (IL)

(73) Assignee: N.M.B. MEDICAL APPLICATIONS LTD., Herzliyail (IL)

(21) Appl. No.: 17/435,706

(22) PCT Filed: Mar. 4, 2020
(86) PCT No.: PCT/IL2020/050248

§ 371 Date: Sep. 2, 2021

Related U.S. Application Data
(60) Provisional application No. 62/813,859, filed on Mar. 5, 2019.

(51) Int. Cl. A61B 3/16; A61B 3/14; A61B 90/00; A61B 3/11

(52) U.S. Cl. CPC A61B 3/165; A61B 3/14; A61B 2090/3937; A61B 3/111; A61B 2090/3904; A61B 90/39

(57) ABSTRACT

A method of non-invasive measurement of an intraocular pressure (IOP), the method may include: obtaining an image of at least a portion of a specific element of a subject's eye; detecting at least a portion of the specific element in the obtained image; and determining a value of a geometric property of the specific element based on the obtained image, the determined value of the geometric property is indicative of an IOP value of the subject's eye. In some embodiments, the specific element comprises at least one of: at least a portion of a limbus and at least a portion of an anterior surface of a sclera of the subject's eye.

### FIELD OF THE INVENTION

The present invention relates to the field of ophthalmology, and more particularly, to non-invasive methods of measurement of an intraocular pressure.

### BACKGROUND OF THE INVENTION

Current techniques of measuring an intraocular pressure (IOP) of a subject typically require direct contact with an eye of the subject. The contact may be achieved either by pressing a sclera of the eye with an applicator (e.g., such as in Goldmann Applanation Tonometer) or by pressing the sclera with an air jet to create local physical depression of the sclera that is further translated into an IOP value. However, such techniques typically require technical skills and cannot be performed by the subject itself.

### SUMMARY OF THE INVENTION

Some embodiments of the present invention may provide a method of non-invasive measurement of an intraocular pressure (IOP), the method may include: obtaining an image of at least a portion of a specific element of a subject's eye, detecting at least a portion of the specific element in the obtained image, and determining a value of a geometric property of the specific element based on the obtained image, the determined value of the geometric property is indicative of an IOP value of the subject's eye, wherein the specific element includes at least one of: at least a portion of a limbus and at least a portion of an anterior surface of a sclera of the subject's eye.

Some embodiments may include receiving: a subject's reference value of the geometric property of the specific element of the subject's eye, the subject's reference value is indicative of a subject's reference IOP value, and determining whether the IOP value of subject's eye is within a predefined range of allowable IOP values from the subject's reference IOP value based on the determined value of the geometric property of the specific element and the subject's reference value of the geometric property thereof.

Some embodiments may include: receiving a subject's reference dataset that includes correlation between IOP values of the subject's eye measured by a clinical IOP measurement device and corresponding values of the geometric property of the specific element of the subject's eye determined based on obtained images, and determining the IOP value of the subject's eye based on the determined value of the geometric property value and the subject's reference dataset.

Some embodiments may include adjusting the determined IOP value based on one or more additional physiological parameter of the subject.

Some embodiments may include notifying at least one of the subject and an authorized third party concerning the determined IOP value of the subject's eye.

Some embodiments may include notifying at least one of the subject and the authorized third party when the determined IOP value of the subject's eye is not within a predefined range of allowable IOP values.

Some embodiments may include controlling a lighting conditions of the subject's eye using light condition control means to ensure that the subject's eye is exposed to substantially the same lighting conditions each time image of the subject's eye being obtained.

Some embodiments may include detecting the specific element of the subject's eye and determining the value of the geometric property thereof based on a selected reference feature of the specific element, the reference feature may include at least one of a specific color and/or specific morphological feature of the specific element.

Some embodiments may include determining the value of the geometric property of the specific element based on a scale marker in the obtained image, wherein the scale marker may include at least one of: a visual marker having known dimensions, the visual marker is applicable to a subject's head in a vicinity of the subject's eye such that the obtained image includes the visual marker, and a predetermined distance between pupils of subject's eye.

Some embodiments may include: storing at least one of: the determined IOP value, the determined value of the value of the geometric property value of the specific element and at least a portion of the obtained image of the subject's eye to generate a record of measurements, analyzing the record of measurements, determining, based on the analysis of thereof, a trend of increase or decrease in determined IOP values, and notifying at least one of the subject and an authorized third party concerning the determined trend of increase or decrease in the determined IOP values.

Some embodiments may include comparing subject's condition to other users based on the record of measurements and at least one of statistical data collected from other users and additional physiological factors of the subject.

Some embodiments of the present invention may provide a device for non-invasive measurement of an intraocular pressure (IOP), the device may include: a camera adapted to obtain an image of at least a portion of a specific element of a subject's eye, and a processing unit configured to detect at least a portion of the specific element in the obtained image, and determine a value of a geometric property of the specific element based on the obtained image, the determined value of the geometric property is indicative of an IOP value of the subject's eye, wherein the specific element may include at least one of: at least a portion of a limbus and at least a portion of an anterior surface of a sclera of the subject's eye.

In some embodiments, the processing unit may be configured to: receive a subject's reference value of the geometric property of the specific element of the subject's eye, the subject's reference value is indicative of a subject's reference IOP value, and determine whether the IOP value of subject's eye is within a predefined range of allowable IOP values from the subject's reference IOP value based on the determined value of the geometric property of the specific element and the subject's reference value of the geometric property thereof.

In some embodiments, the processing unit may be configured to: receive a subject's reference dataset that includes correlation between IOP values of the subject's eye measured by a clinical IOP measurement device and corresponding values of the geometric property of the specific element of the subject's eye determined based on obtained images, and determine the IOP value of the subject's eye based on the determined value of the geometric property value and the subject's reference dataset.

In some embodiments, the processing unit may be configured to adjust the determined IOP value based on one or more additional physiological parameter of the subject.

In some embodiments, the device may include a notification unit configured to notify at least one of the subject and an authorized third party concerning the determined IOP value of the subject's eye.

In some embodiments, the device may include a notification unit configured to notify at least one of the subject and the authorized third party when the determined IOP value of the subject's eye is not within a predefined range of allowable IOP values.

In some embodiments, the device may include light condition control means configured to ensure that the subject's eye is exposed to substantially the same lighting conditions each time image of the device being used.

In some embodiments, the processing unit may be configured to detect the specific element of the subject's eye and determining the value of the geometric property thereof based on a selected reference feature of the specific element, the reference feature may include at least one of a specific color and/or specific morphological feature of the specific element.

In some embodiments, the processing unit may be configured to determine the value of the geometric property of the specific element based on a scale marker in the obtained image, wherein the scale marker may include at least one of: a visual marker having known dimensions, the visual marker is applicable to a subject's head in a vicinity of the subject's eye such that the obtained image includes the visual marker, and a predetermined distance between pupils of subject's eye.

In some embodiments, the device may include a memory configured to store at least one of: the determined IOP value, the determined value of the value of the geometric property value of the specific element and at least a portion of the obtained image of the subject's eye to generate a record of measurements.

In some embodiments, the processing unit may be configured to analyze the record of measurements and determine, based on the analysis of thereof, a trend of increase or decrease in determined IOP values.

In some embodiments, the device may include a notification unit configured to notify at least one of the subject and an authorized third party concerning the determined trend of increase or decrease in the determined IOP values.

In some embodiments, the processing unit may be configured to compare subject's condition to other users based on the record of measurements and at least one of statistical data collected from other users and additional physiological factors of the subject.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same can be carried into effect, reference will now be made, purely by way of example, to the accompanying drawings in which like numerals designate corresponding elements or sections throughout.

In the accompanying drawings:

FIG. 1 is a schematic block diagram of a device for non-invasive measurement of an intraocular pressure (IOP), according to some embodiments of the invention;

FIG. 2 is a flowchart of a method of non-invasive measurement of an intraocular pressure (IOP), according to some embodiments of the invention;

FIG. 3A depicts an image of a pig's eye enhanced with a naso-temporal diameter and a vertical diameter of a limbus thereof determined according to some embodiments of the invention;

FIG. 3B depicts a processed image of a portion of a limbus of a pig's eye enhanced with a naso-temporal line tangent to the limbus and determined based on a selected reference color according to some embodiments of the invention;

FIG. 3C depicts a processed image of a portion of limbus of a pig's eye enhanced with a vertical line tangent to the limbus and determined based on a selected reference color according to some embodiments of the invention;

FIGS. 3D and 3E depict results showing a change of a naso-temporal diameter and a vertical diameter of a limbus of a pig's eye, respectively, determined according to some embodiments of the invention, in response to change of an IOP in the pig's eye; and

FIG. 4 depicts a table showing results of in-vivo experiment of non-invasive measurement of an intraocular pressure (IOP) in human, according to some embodiments of the invention.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention can be practiced without the specific details presented herein. Furthermore, well known features can have been omitted or simplified in order not to obscure the present invention. With specific reference to the drawings, it is stressed that the particulars shown are by way of example and for purposes of illustrative discussion of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention can be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that can be practiced or carried out in various ways as well as to combinations of the disclosed embodiments. Also, it is to be understood that the phraseology and terminology employed herein is for the purpose of description and should not be regarded as limiting.

Unless specifically stated otherwise, as apparent from the following discussions, it is appreciated that throughout the specification discussions utilizing terms such as “processing”, “computing”, “calculating”, “determining”, “enhancing” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices. Any of the disclosed modules or units can be at least partially implemented by a computer processor.

Some embodiments of the present invention provide a method and a device for non-invasive measurement of an intraocular pressure (IOP).

It has been found by the inventors that a change in at least some geometric properties of a limbus and/or of an anterior surface of a sclera of the subject's eye may be indicative of a change of an IOP value of the subject's eye with respect to a reference IOP value thereof. The geometric properties may, for example, include at least one of: a diameter of the limbus along at least one of reference axes thereof, a circumference of the limbus, an area enclosed by the limbus, a curvature of an anterior surface of the sclera and/or any derivation thereof and/or any combination thereof.

Some embodiments may include obtaining an image of at least a portion of a specific element of the subject's eye (e.g., the limbus and/or the anterior surface of the sclera), detecting at least a portion of the specific element in the obtained image, and determining a value of a geometric property of the specific element (e.g., the diameters(s), the circumference, the area enclosed by the limbus and/or the curvature of the anterior surface of the sclera) based on the obtained image, wherein the determined value of the geometric property value is indicative of an IOP value of the subject's eye.

Various embodiments may further include determining whether the IOP value of the subject's eye is within a predefined range from a subject's reference IOP value and/or determining the IOP value of the subject's eye based on the determined geometric property value and a subject's reference dataset.

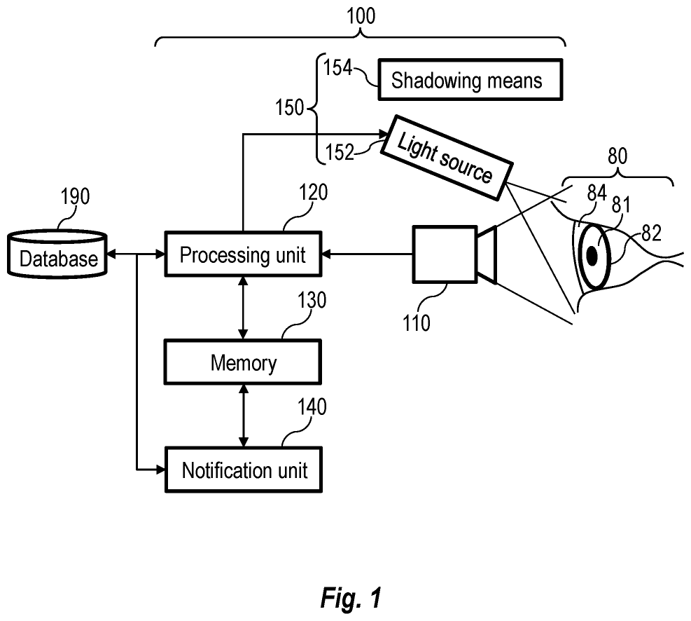

The disclosed invention may be implemented on a dedicated device (e.g., such as device 100 described below with respect to FIG. 1) or using a software application executable on, for example, a portable computing device (e.g., a smartphone, a tablet, etc.) of the subject. Advantageously, utilization of the device/application does not require any special technical skills. Accordingly, the measurement of the IOP may be performed by the subject itself, for example, at subject's home, without a need in visiting an ophthalmologist or a skilled technician. For example, the subject may be required to visit the ophthalmologist or the skilled technician prior to first use of the device/application and/or few more times during the use of the device/application for generation of the subject's reference dataset and perform the rest of the measurements by itself, e.g., at subject's home. The device/application may enable the subject to measure the IOP on a daily basis, or several times a day, according to ophthalmologist's instructions and subject's condition, while reducing the frequency of clinic IOP measurements to minimum. In some embodiments, the device/application may be implemented for home screening of healthy population. For example, an individual may monitor it's IOP using the device/application and the device/application may be configured to alert for any abnormal change compared to the individual's history, and possibly, recommend medical attention if needed.

Reference is now made to FIG. 1, which is a schematic block diagram of a device 100 for non-invasive measurement of an intraocular pressure (IOP), according to some embodiments of the invention.

According to some embodiments, device 100 may include an optical sensor (herein after a camera) 110 and a processing unit 120.

Camera 110 may obtain an image of at least a portion of a specific element of a subject's eye 80. In various embodiments, the image may include one eye or both eyes of the subject. Processing unit 120 may detect at least a portion of the specific element in the obtained image. Processing unit 120 may determine a value of a geometric property of the specific element based on the obtained image, wherein the determined value of the geometric property is indicative of an IOP of subject's eye 80.

In some embodiments, the specific element may be at least a portion of a limbus 82 of subject's eye 80. Limbus 82 is an intermediate region between a cornea 81 and a sclera 84 of subject's eye 80. In some embodiments, the specific elements may be at least a portion of an anterior surface of sclera 84. In some embodiments, the geometric property of limbus 82 may include at least one of the following parameters: a naso-temporal diameter of limbus 82, a vertical diameter of limbus 82, a circumference of limbus 82, an area enclosed by limbus 82 and a curvature of the anterior surface of sclera 84. In some embodiments, the geometric property may be a derivation of at least one of the parameters thereof. For example, the geometric property may be a ratio between the naso-temporal diameter and the vertical diameter. Other derivations and/or combinations of the values are also possible.

In some embodiments, device 100 may include a memory 130. Memory 130 may store at least a subject's reference values of the geometric property of the specific element of subject's eye 80 that is indicative of a subject's reference IOP value.

The correlation between the subject's reference IOP value and the subject's reference value of the geometric property of the specific element may be predetermined by, for example, an authorized authority (e.g., a skilled technician, ophthalmologist, etc.), prior to first use of the device. For example, the authorized authority may measure the subject's reference IOP value with a clinical IOP measurement device (e.g., such as Goldmann Applanation Tonometer, etc.) and determine the subject's reference value of the geometric property of the specific element using device 100, and store the correlation therebetween in memory 130 of device 100.

In some embodiments, processing unit 120 may receive the subject's reference value of the geometric property of the specific element from memory 130 and determine whether the IOP value of subject's eye is within a predefined range of allowable IOP values from the subject's reference IOP value based on the determined value of the geometric property of the specific element and the subject's reference value of the geometric property thereof.

In some embodiments, memory 130 may store a subject's reference dataset that may include correlation between IOP values of the subject's eye measured by the clinical IOP measurement device and corresponding values of the geometric property of the specific element of the subject's eye determined by device 100. The subject's reference dataset may be generated by, for example, the authorized authority (e.g., as described above). For example, the subject may go through several follow up measurement of the IOP values and corresponding values of the geometric property of the specific element to build the subject's reference dataset.

For example, Equation 1 provides an example of a relation between the currently determined IOP value (IOP), currently determined value of the geometric property of the specific element (V_{gp}), predetermined set of IOP values (IOPₚᵣₑₛₑₜ), predetermined set of values of the geometric property of the specific element (V_{gp_preset}) and optionally physiological parameters of the subject (Physₚₐᵣ), light conditions (L_{con}), etc.:

IOP=f(V_{gp}, IOPₚᵣₑₛₑₜ, V_{gp_preset}, Physₚₐᵣ, L_{con}) (Equation 1)

In some embodiments, processing unit 120 may determine the IOP value of the subject's eye based on the determined value of the geometric property and the subject's reference dataset. The determined IOP value may, for example, be an absolute number ranging between 10-50 mmHg (e.g., between 10-20 mmHg, 10-25 mmHg, etc.).

In some embodiments, processing unit 120 may adjust the determined IOP value based on one or more additional physiological parameters of the subject. The physiological parameter(s) may, for example, include a blood pressure, a heartrate, an age, a gender, a medical condition of the subject, etc.

In some embodiments, memory 130 may store at least one of: the determined IOP value, the determined value of the geometric property of the specific element and at least a portion of the obtained image of subject's eye 80 to generate a record of measurements.

In some embodiments, device 100 may include a notification unit 140. Notification unit 140 may notify the subject and/or an authorized third party concerning the determined IOP value of subject's eye 80. For example, notification unit 140 may include a display for displaying the determined IOP value to the subject. Notification unit 140 may, for example, include a communication sub-unit for sending the determined IOP value of subject's eye 80 to the authorized third party (e.g., subjects' ophthalmologist, etc.). In various embodiments, notification unit 140 may notify the subject and/or the authorized third party when the determined IOP value of subject's eye 80 is not within a predefined range of allowable IOP values. The range of allowable IOP values may be predefined by, for example, the authorized third party and stored in memory 130 of device 100.

In some embodiments, processing unit 120 may analyze the record of measurements. Processing unit 120 may determine, based on the analysis of the record of measurements, a trend of increase or decrease in determined IOP values. Notification unit 140 may, for example, notify the subject and/or the authorized third party concerning the determined trend thereof.

In some embodiments, processing unit 120 may be in communication with a database 190. Database 190 may include statistical data collected from, for example, multiple users. Processing unit 120 may compare subject's condition to other users based on the subject's record of measurements and the statistical data and/or additional factors such as age, gender, race, blood pressure, any eye disease, etc. of the subject. Notification unit 140 may, for example, notify the subject and/or the authorized authority concerning the outcomes of the comparison thereof.

In some embodiments, device 100 may include light condition control means 150. Light condition control means 150 may be configured to ensure that subject's eye 80 is exposed to the same lighting conditions (or substantially the same lighting conditions) each time device 100 is used. For example, light condition control means 150 may include at least one of: a controllable light source 152 and shadowing means 154. At least controllable light source 152 may be controlled by processing unit 120, for example, based on the image being obtained by camera 110. In some embodiments, notification unit 140 may advise the subject of poor frame obtaining conditions and guide the subject to reach better frame obtaining conditions, e.g., set lighting and or shading conditions, change of relative angle of image with regard to the camera, and the like.

In some embodiments, processing unit 120 may detect the specific element of subject's eye 80 and determine the value of the geometric property thereof based on a selected reference feature of the specific element. The reference feature of the specific element of subject's eye 80 may, for example, include a specific color and/or morphological feature of the specific element. The reference feature may be selected by, for example, the authorized authority (e.g., ophthalmologist) during reference measurements of IOP values and values of the geometric property of the specific element. Selection of the reference feature may, for example, ensure repetitive and precise (or optimized precision) detection of the specific element of subject's eye 80 in the obtained images and repetitive and/or precise determination of the value of the geometric property thereof based on the obtained images.

For example, in case of limbus 82, a reference color (or a reference greyscale level) may be selected as the reference feature. In this example, the obtained image may be processed to detect pixels containing the reference color to identify, or determine, the circumference line of limbus 82 within optionally more than one pixel residing in the transient region between the cornea and the sclera regions.

In another example, in case of the anterior surface of sclera 84, an angle of reflection of light from a predefined point on the anterior surface of sclera 84 may be selected as the reference feature. In this example, a change in the reflection angle of light from the predefined point on the anterior surface of sclera 84 may be indicative of a change of the curvature of thereof.

In some embodiments, processing unit 120 may utilize a sensing technology to identify micro-level changes in the determined values of the geometric property of the specific element of subject's eye 80. For example, an optical focus within sclera 84 and/or the anterior surface of sclera 84 may be identified, or determined, using an ultrasonic-based device and/or a laser doppler device.

In some embodiments, processing unit 120 may determine the value of the geometric property of the specific element based on a scale marker in the obtained image. The scale marker may, for example, include a visual marker having known dimensions. The visual marker may be applicable to the subject's head in a vicinity of subject's eye(s) such that the obtained image includes the visual marker when captured. In another example, the scale marker may, for example, be a visual physiological marker of the subject. One example of the visual physiological marker may include a predetermined distance between pupils of the subject's eyes, which has a constant value for each subject. The distance between the pupils of the subject's eyes may be predetermined (e.g., premeasured) by, for example, the authorized authority (e.g., ophthalmologist) during reference measurements of IOP values and values of the geometric property of the specific element.

In some embodiments, at least some units of device 100 may be implemented on a portable computing device (e.g., tablet, smartphone, etc.).

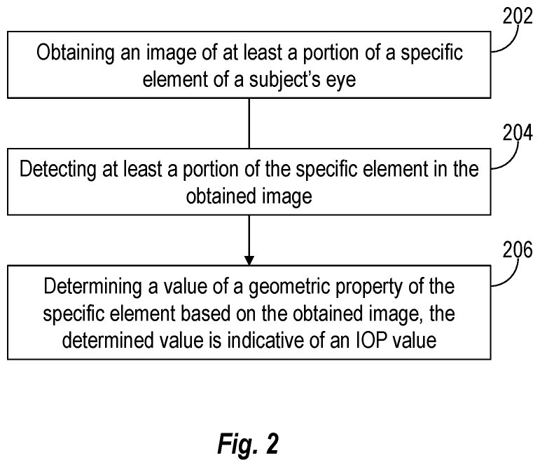

Reference is now made to FIG. 2, which is a flowchart of a method of non-invasive measurement of an intraocular pressure (IOP), according to some embodiments of the invention.

The method may be implemented by a device for non-invasive IOP measurement (e.g., such as device 100 described above with respect to FIG. 1), which may be configured to implement the method. It is noted that the method is not limited to the flowcharts illustrated in FIG. 2 and to the corresponding description. For example, in various embodiments, the method needs not move through each illustrated box or stage, or in exactly the same order as illustrated and described.

Some embodiments may include obtaining an image of at least a portion of a specific element of a subject's eye (stage 202). For example, as described above with respect to FIG. 1. In various embodiments, the image may include one eye or both eyes of the subject.

Some embodiments may include detecting at least a portion of the specific element in the obtained image (stage 204). For example, using processing unit 120 as described above with respect to FIG. 1. The specific element may, for example, be at least a portion of a limbus or at least a portion of an anterior surface of a sclera of the subject's eye (e.g., as described above with respect to FIG. 1).

Some embodiments may include determining a value of a geometric property of the specific element based on the obtained image, wherein the determined value of the geometric property is indicative of an IOP value of the subject's eye (stage 206). For example, as described above with respect to FIG. 1.

The geometric property of the limbus may, for example, include at least one of the following parameters: a naso-temporal diameter of limbus, a vertical diameter of limbus, a circumference of limbus, an area enclosed by limbus and a curvature of the anterior surface of sclera. In another example, the geometric property may be a derivation of at least one of the parameters. For example, the geometric property may be a ratio between the naso-temporal diameter and the vertical diameter. Other derivations and/or combinations of the values are also possible. (e.g., as described above with respect to FIG. 1)

Some embodiments may include detecting the specific element of the subject's eye and determining the value of the geometric property thereof based on a selected reference feature of the specific element. The reference feature of the specific element of the subject's eye may, for example, include a specific color and/or specific morphological feature of the specific element. (e.g., as described above with respect to FIG. 1)

Some embodiments may include determining the value of the geometric property of the specific element based on a scale marker in the obtained image. The scale marker may, for example, include a visual marker having known dimensions. The visual marker may be applicable to the subject's head in a vicinity of subject's eye(s) such that the obtained image includes the visual marker when captured. In another example, the scale marker may, for example, be a visual physiological marker of the subject. One example of the visual physiological marker may include a predetermined distance between pupils of the subject's eyes, which has a constant value for each subject. (e.g., as described above with respect to FIG. 1)

Some embodiments may include receiving a subject's reference value of the geometric property of the specific element of the subject's eye that is indicative of a subject's reference IOP value. For example, as described above with respect to FIG. 1.

The correlation between the subject's reference IOP value and the subject's reference value of the geometric property of the specific element may be predetermined by, for example, an authorized authority (e.g., a skilled technician, ophthalmologist, etc.) prior to first use of the device. For example, the authorized authority may measure the subject's reference IOP value with a clinical IOP measurement device (e.g., such as Goldmann Applanation Tonometer, etc.) and determine the subject's reference value of the geometric property of the specific element by processing the obtained image (e.g., as described above) to determine the correlation therebetween.

Some embodiments may include determining whether the IOP value of subject's eye is within a predefined range of allowable IOP values from the subject's reference IOP value based on the determined value of the geometric property of the specific element and the subject's reference value of the geometric property thereof. For example, as described above with respect to FIG. 1.

For example, current determined IOP value (IOP) of a given subject may be expressed by Equation 2:

IOP=A·x+B (Equation 2)

wherein A may define the relation between the changing determined value (x) and the change in determined IOP value and B may define the offset of the IOP value from a reference averaged set obtained from a large number of objects. Optionally, parameters A and B can be determined by obtaining more than one measurement of IOP and (x). Optionally, nonlinear function may be used. Optionally, more than one geometric parameter may be used as an input for the IOP calculation, for example average of the IOP calculated based on naso-temporal diameter measurement and the IOP calculated based on vertical diameter measurement of IOP. It would be apparent to those skilled in the art that other mathematical relations may be used in determining the IOP.

Some embodiments may include receiving a subject's reference dataset that includes correlation between IOP values of the subject's eye measured by a clinical IOP measurement device and corresponding values of the geometric property of the specific element of the subject's eye determined based on processing of the obtained images. For example, as described above with respect to FIG. 1.

Some embodiments may include determining the IOP value of the subject's eye based on the determined value of the geometric property value and the subject's reference dataset. For example, as described above with respect to FIG. 1.

Some embodiments may include adjusting the determined IOP value based on one or more additional physiological parameter of the subject. For example, as described above with respect to FIG. 1. The physiological parameter(s) may, for example, include a blood pressure, a heartrate, an age, a gender, a medical condition of the subject, etc. (e.g., as described above with respect to FIG. 1).

Some embodiments may include storing at least one of: the determined IOP value, the determined value of the value of the geometric property value of the specific element and at least a portion of the obtained image of the subject's eye to generate a record of measurements. For example, as described above with respect to FIG. 1.

Some embodiments may include notifying the subject and/or an authorized third party concerning the determined IOP value of the subject's eye. For example, as described above with respect to FIG. 1.

Some embodiments may include notifying the subject and/or the authorized third party when the determined IOP value of the subject's eye is not within a predefined range of allowable IOP values. For example, as described above with respect to FIG. 1.

Some embodiments may include analyzing the record of measurements and determining, based on the analysis of the record of measurements, a trend of increase or decrease in determined IOP values. For example, as described above with respect to FIG. 1.

Some embodiments may include notifying the subject and/or the authorized third party concerning the determined trend of increase or decrease in determined IOP values. For example, as described above with respect to FIG. 1.

Some embodiments may include comparing subject's condition to other users based on the record of measurements and statistical data collected from other users and/or additional physiological factors of the subject such as age, gender, race, blood pressure, any eye disease, etc. Some embodiments may include notifying at least one of the subject and the authorized authority concerning outcomes of the comparison thereof. For example, as described above with respect to FIG. 1.

Some embodiments may include controlling a lighting conditions of the subject's eye using light condition control means. For example, as described above with respect to FIG. 1. This may, for example, ensure that the subject's eye is exposed to the same lighting conditions (or substantially the same lighting conditions) each time the image of the subject's eye being obtained.

Some embodiments may include advising the subject of poor frame obtaining conditions and guide the subject to reach better frame obtaining conditions, e.g., set lighting and or shading conditions, change of relative angle of image with regard to the camera, and the like.

Some embodiments may include implementing at least a portion of the method by a portable computing device (e.g., tablet, smartphone, etc.). For example, as described above with respect to FIG. 1.

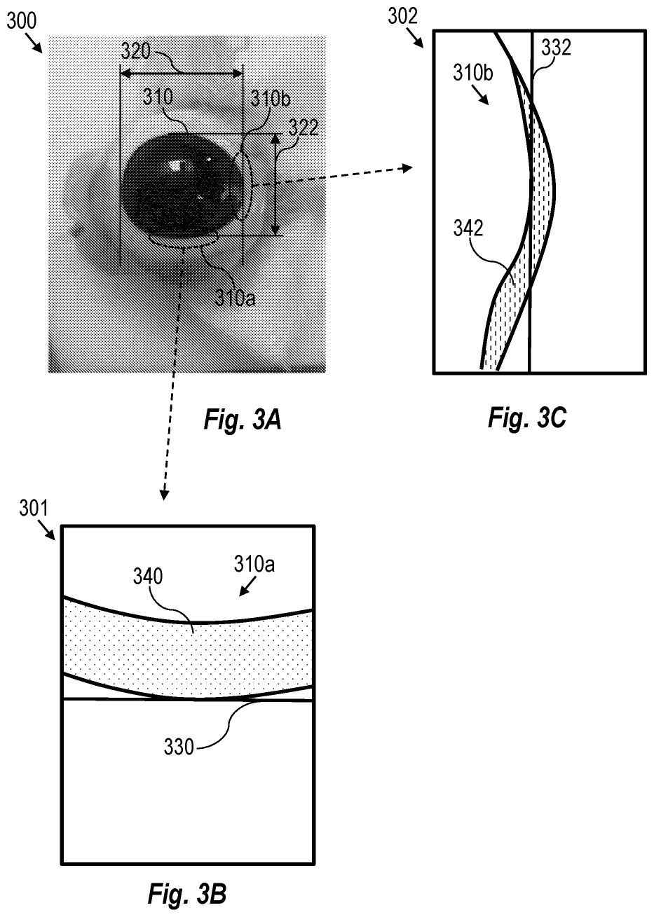

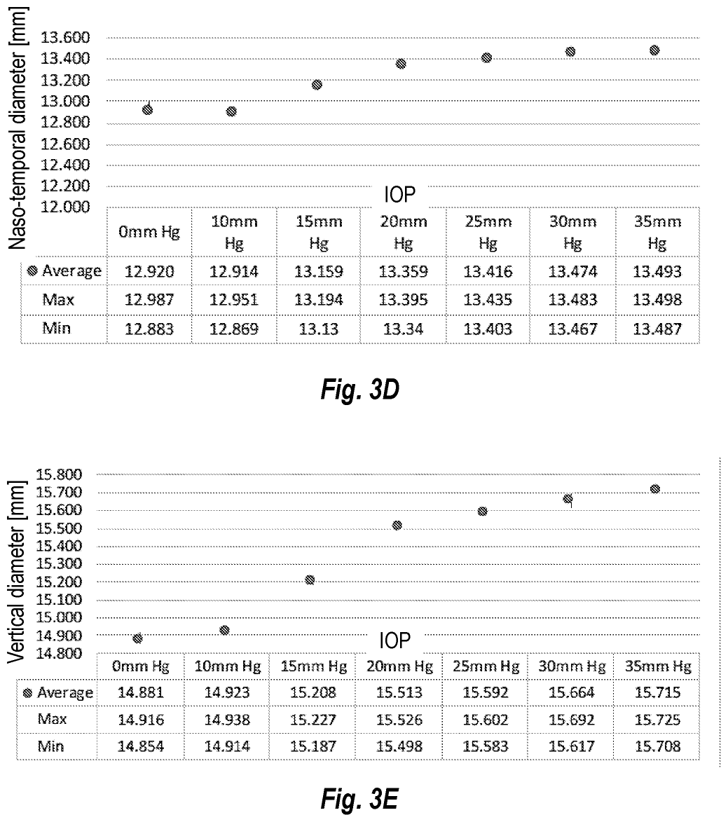

Reference is now collectively made to FIGS. 3A, 3B, 3C, 3D and 3E depicting results of an ex-vivo experiment of non-invasive measurement of an intraocular pressure (IOP) in a pig's eye, according to some embodiments of the invention.

FIG. 3A depicts an image 300 of a pig's eye enhanced with a naso-temporal diameter 320 and a vertical diameter 322 of a limbus 310 thereof determined according to some embodiments of the invention.

In some embodiments, limbus 310 may be detected in image 300 based on a selected reference feature of limbus 310. For example, the selected reference features may be a selected reference color of limbus 310. For example, image 300 may be processed to detect pixels containing the selected reference color to identify the circumference of limbus 310. The selected reference color may, for example, be used to ensure repetitive and precise detection of limbus 310 in obtained images (e.g., like image 300) of the eye and repetitive and/or precise determination of the value of the geometric property thereof based on the obtained images.

For example, FIG. 3B depicts a processed image 301 of a portion 310a of limbus 310 of the pig's eye enhanced with a naso-temporal line 330 tangent to limbus 310 and determined based on a selected reference color 340 according to some embodiments of the invention. FIG. 3C depicts a processed image 302 of a portion 310b of limbus 310 of the pig's eye enhanced with a vertical line 332 tangent to limbus 310 and determined based on a selected reference color 342 according to some embodiments of the invention.

It has been found by the inventors that a change in at least some geometric properties of limbus 310 of the pig's eye may be indicative of a change of an IOP value of the pig's eye with respect to a reference IOP value thereof. For example, FIGS. 3D and 3E depict results showing a change of a naso-temporal diameter and a vertical diameter of the limbus of the pig's eye, respectively, determined according to some embodiments of the invention, in response to change of the IOP in the pig's eye (e.g., such as naso-temporal diameter 320 and vertical diameter 322 shown in FIG. 3A, respectively).

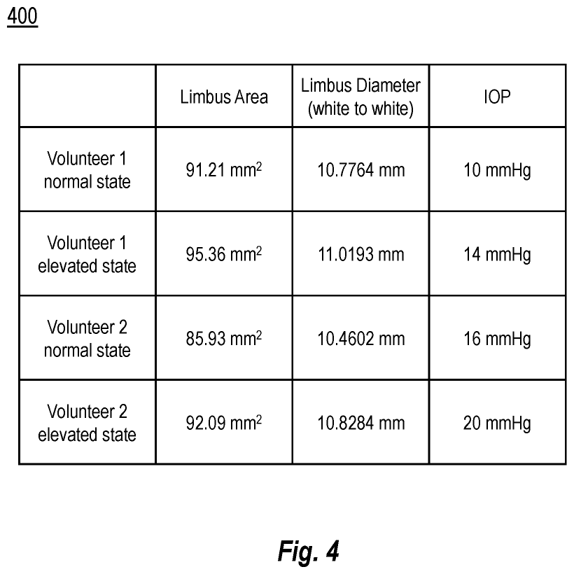

Reference is now made to FIG. 4, which depicts a table 400 showing results of in-vivo experiment of non-invasive measurement of an intraocular pressure (IOP) in human, according to some embodiments of the invention.

The experiment has been performed in a controlled environment with a consistent light source. Two volunteers (e.g., referred hereinafter as “volunteer 1” and “volunteer 2”) participated in the experiment.

At the first stage, the volunteer sat in a designated chair for 5 minutes to make sure that its baseline IOP is at its normal values. The IOP value has been measured with a clinical iCare device and an image of the volunteer's eye has been obtained using a camera. The image of the volunteers' eyes has been analyzed using a Jimage application and a diameter (e.g., white-to-white distance) and an area of the limbus have been calculated. The values are presented in Table 400 as “volunteer 1 normal state” and “volunteer 2 normal state”.

At the next stage, the volunteer has been asked to blow in a designated tube with an exit way of 22 gauge and to produce a pressure of 40 mmHg for 20 seconds. After 15 seconds into the blowing process another image of the volunteer's eye has been obtained, followed by a second IOP measure with the clinical iCare device. The image of the volunteers' eyes has been analyzed and the diameter and the area of the limbus have been calculated. The values are presented in Table 400 as “volunteer 1 elevated state” and “volunteer 2 elevated state”.

The experiment showed a change of 4.55% and 7.12% in the limbus area corresponding to a change of 40% and 25% in the measured IOP value between the normal state and the elevated state for volunteer 1 and volunteer 2, respectively. The experiment also showed a change of 2.25% and 3.52% in the limbus diameter corresponding to a change of 40% and 25% in the measured IOP value between the normal state and the elevated state for volunteer 1 and volunteer 2, respectively.

The disclosed invention may be implemented on a dedicated device (e.g., such as device 100 described below with respect to FIG. 1) or using a software application executable on, for example, a portable computing device (e.g., a smartphone, a tablet, etc.) of the subject. Advantageously, utilization of the device/application does not require any special technical skills. Accordingly, the measurement of the IOP may be performed by the subject itself, for example, at subject's home, without a need in visiting an ophthalmologist or a skilled technician. For example, the subject may be required to visit the ophthalmologist or the skilled technician prior to first use of the device/application and/or few more time during the use of the device/application for generation of the subject's reference dataset and perform the rest of the measurements by itself, e.g., at subjet's home. The device/application may enable the subject to measure the IOP on a daily basis, or several times a day, according to ophthalmologist's instructions and subject's condition, while reducing the frequency of clinic IOP measurements to minimum. In some embodiments, the device/application may be implemented for home screening of healthy population. For example, an individual may monitor it's IOP using the device/application and the device/application may be configured to alert for any abnormal change compared to the individual's history, and possibly, recommend medical attention if needed.

Aspects of the present invention are described above with reference to flowchart illustrations and/or portion diagrams of methods, apparatus (systems) and computer program products according to embodiments of the invention. It will be understood that each portion of the flowchart illustrations and/or portion diagrams, and combinations of portions in the flowchart illustrations and/or portion diagrams, can be implemented by computer program instructions. These computer program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or portion diagram or portions thereof.

These computer program instructions can also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or portion diagram portion or portions thereof. The computer program instructions can also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or portion diagram portion or portions thereof.

The aforementioned flowchart and diagrams illustrate the architecture, functionality, and operation of possible implementations of systems, methods and computer program products according to various embodiments of the present invention. In this regard, each portion in the flowchart or portion diagrams can represent a module, segment, or portion of code, which includes one or more executable instructions for implementing the specified logical function(s). It should also be noted that, in some alternative implementations, the functions noted in the portion can occur out of the order noted in the figures. For example, two portions shown in succession can, in fact, be executed substantially concurrently, or the portions can sometimes be executed in the reverse order, depending upon the functionality involved. It will also be noted that each portion of the portion diagrams and/or flowchart illustration, and combinations of portions in the portion diagrams and/or flowchart illustration, can be implemented by special purpose hardware-based systems that perform the specified functions or acts, or combinations of special purpose hardware and computer instructions.

In the above description, an embodiment is an example or implementation of the invention. The various appearances of “one embodiment”, “an embodiment”, “certain embodiments” or “some embodiments” do not necessarily all refer to the same embodiments. Although various features of the invention can be described in the context of a single embodiment, the features can also be provided separately or in any suitable combination. Conversely, although the invention can be described herein in the context of separate embodiments for clarity, the invention can also be implemented in a single embodiment. Certain embodiments of the invention can include features from different embodiments disclosed above, and certain embodiments can incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A method of non-invasive measurement of an intraocular pressure (IOP), the method comprising:
obtaining an image of at least a portion of a specific element of a subject's eye;
detecting at least a portion of the specific element in the obtained image; and
determining a value of a geometric property of the specific element based on the obtained image, the determined value of the geometric property is indicative of an IOP value of the subject's eye;
wherein the specific element comprises at least one of at least a portion of a limbos and at least a portion of an anterior surface of a sclera of the subject's eye.

2. The method of claim 1, comprising:
receiving a subject's reference value of the geometric property of the specific element of the subject's eve, the subject's reference value is indicative of a subject's reference IOP value; and
determining whether the IOP value of subject's eye is within a predefined range of allowable IOP values from the subject's reference IOP value based on the determined value of the geometric property of the specific element and the subject's reference value of the geometric property thereof.

3. The method of claim 1, comprising:
receiving a subject's reference dataset that includes correlation between IOP values of the subject's eye measured by a clinical IOP measurement device and corresponding values of the geometric property of the specific element of the subject's eye determined based on obtained images; and
determining the IOP value of the subject's eye based on the determined value of the geometric property value and the subject's reference dataset.

4. The method of claim 3, comprising adjusting the determined IOP value based on one or more additional physiological parameter of the subject.

5. The method of claim 3, comprising notifying at least one of the subject and an authorized third party concerning the determined IOP value of the subject's eye.

6. The method of claim 3. comprising notifying at least one of the subject and the authorized third party when the determined IOP value of the subject's eye is not within a predefined range of allowable IOP values.

7. The method of claim 3, comprising controlling a lighting conditions of the subject's eye using light condition control means to ensure that the subject's eye is exposed to substantially the same lighting conditions each time image of the subject's eye being obtained.

8. The method of claim 1, comprising detecting the specific element of the subject's eye and determining the value of the geometric property thereof based on a selected reference feature of the specific element, the reference feature comprises at least one of a specific color and/or specific morphological feature of the specific element.

9. The method of claim 1, comprising determining the value of the geometric property of the specific element based on a scale marker in the obtained image; wherein the scale marker comprises at least one of: a visual marker having known dimensions, the visual marker is applicable to a subject's head in a vicinity of the subject's eye such that the obtained image includes the visual marker, and a predetermined distance between pupils of subject's eye.

10. The method of claim 1, comprising:
storing at least one of: the determined IOP value, the determined value of the value of the geometric property value of the specific element and at least a portion of the obtained image of the subject's eye to generate a record of measurements;
analyzing the record of measurements;
determining, based on the analysis of thereof, a trend of increase or decrease in determined IOP values; and
notifying at least one of the subject and an authorized third party concerning the determined trend of increase or decrease in the determined IOP values.

11. (canceled)

12. A device for non-invasive measurement of an intraocular pressure (IOP), the device comprising:
a camera adapted to obtain an image of at least a portion of a specific element of a subject's eye; and
a processing unit configured to:
detect at least a portion of the specific element in the obtained image; and
determine a value of a geometric property of the specific element based on the obtained image, the determined value of the geometric property is indicative of an IOP value of the subject's eye;
wherein the specific element comprises at least one of at least a portion of a limbus and at least a portion of an anterior surface of a sclera of the subject's eye.

13. The device of claim 12, wherein the processing unit is configured to:
receive a subject's reference value of the geometric property of the specific element of the subject's eye, the subject's reference value is indicative of a subject's reference IOP value; and
determine whether the IOP value of subject's eye is within a predefined range of allowable IOP values from the subject's reference IOP value based on the determined value of the geometric property of the specific element and the subject's reference value of the geometric property thereof.

14. The device of claim 12, wherein the processing unit is configured to:
receive a subject's reference dataset that includes correlation between IOP values of the subject's eye measured by a clinical IOP measurement device and corresponding values of the geometric property of the specific element of the subject's eye determined based on obtained images; and
determine the IOP value of the subject's eve based on the determined value of the geometric property value and the subject's reference dataset.

15. The device of claim 14, wherein the processing unit is configured to adjust the determined IOP value based on one or more additional physiological parameter of the subject.

16. The device of claim 14, comprising a notification unit configured to notify at least one of the subject and an authorized third party concerning the determined IOP value of the subject's eye.

17. The device of claim 14, comprising a notification unit configured to notify at least one of the subject and the authorized third party when the determined IOP value of the subject's eye is not within a predefined range of allowable IOP values.

18. The device of claim 12, comprising light condition control means configured to ensure that the subject's eye is exposed to substantially the same lighting conditions each time image of the device being used.

19. The device of claim 12, wherein the processing unit is configured to detect the specific element of the subject's eye and determining the value of the geometric property thereof based on a selected reference feature of the specific element, the reference feature comprises at least one of a specific color and/or specific morphological feature of the specific element.

20. The device of claim 12, wherein the processing unit is configured to determine the value of the geometric property of the specific element based on a scale marker in the obtained image, and wherein the scale marker comprises at least one of a visual marker having known dimensions, the visual marker is applicable to a subject's head in a vicinity of the subject's eye such that the obtained image includes the visual marker, and a predetermined distance between pupils of subject's eye.

21. The device of claim 12:
comprising a memory configured to store at least one of: the determined IOP value, the determined value of the value of the geometric property value of the specific element and at least a portion of the obtained image of the subject's eye to generate a record of measurements;
wherein the processing unit is configured to analyze the record of measurements and determine, based on the analysis of thereof, a trend of increase or decrease in determined IOP values; and
comprising a notification unit configured to notify at least one of the subject and an authorized third party concerning the determined trend of increase or decrease in the determined IOP values.

22. (canceled)
